(19) United States
(12) Patent Application Publication
(10) Pub. No.: US 2023/0392953 A1
(43) Pub. Date: Dec. 7, 2023

(54) STRIDE LENGTH ESTIMATION AND CALIBRATION AT THE WRIST

(71) Applicant: Apple Inc., Cupertino, CA (US)

(72) Inventors: Lucie A. Huet, Mountain View, CA (US); Adeeti V. Ullal, Emerald Hills, CA (US); Allison L. Gilmore, Redwood City, CA (US); Gabriel A. Blanco, San Francisco, CA (US); Karthik Jayaraman Raghuram, Foster City, CA (US); Maryam Etezadi-Amoli, Santa Clara, CA (US); Richard A. Fineman, Campbell, CA (US)

(21) Appl. No.: 18/205,478

(22) Filed: Jun. 2, 2023

Related U.S. Application Data
(60) Provisional application No. 63/349,091, filed on Jun. 4, 2022.

(51) Int. Cl. G01C 22/00; A61B 5/11; A61B 5/00; G01C 25/00

(52) U.S. Cl. CPC G01C 22/006; A61B 5/112; A61B 5/681; G01C 25/00; A61B 2562/0219

(57) ABSTRACT

Embodiments are disclosed for stride length estimation and calibration at the wrist. In some embodiments, a method comprises: obtaining sensor data from a wearable device worn on a wrist of a user; deriving features from the sensor data; estimating a form-based stride length using an estimation model that takes the features and user height as input; and calibrating the form-based stride length. In other embodiments, user cadence and speed are used to estimate speed-based stride length which, upon certain conditions, is blended with the form-based stride length to get a final estimated stride length of the user.

### CROSS-REFERENCE TO RELATED APPLICATION

This application claims priority to U.S. Provisional Patent Application No. 63/349,091, filed Jun. 4, 2022, the entire contents of which are incorporated herein by reference.

### TECHNICAL FIELD

This disclosure relates generally to health monitoring and fitness applications.

### BACKGROUND

Stride length (also referred to as step length) is the distance from the heel print of one foot to the heel print of the other foot during a walking/running stride. A fitness application can multiply stride length by a step count from a digital pedometer to compute distance traveled by a runner. Stride length depends on height. The average stride length for a man is 2.5 feet and the average stride length for women is 2.2 feet. A rough estimate of stride length can be computed by multiplying height by a gender-based scale factor to get stride length. For example, height can be multiplied by 0.413 to get stride length (in inches) for a female and multiplied by 0.415 to get stride length for a male. In many fitness applications, it is desirable to have a more personalized estimate of stride length to ensure that fitness metrics that utilize stride length as a factor are more accurate, such as distance traveled.

### SUMMARY

Embodiments are disclosed for estimating stride length at the wrist. In some embodiments, a method comprises: obtaining, with at least one processor, sensor data from a wearable device worn on a wrist of a user; deriving, with the at least one processor, features from the sensor data; estimating, with the at least one processor, stride length using an estimation model that takes the features and user height as input; and calibrating, with the at least one processor, the stride length.

In some embodiments, the sensor data includes acceleration and rotation rate, and the features include at least one of square root of the mean of transverse acceleration, maximum vertical rotation rate or minimum normalized rotation rate.

In some embodiments, calibrating comprises calculating a bias offset using distances from calibration tracks and stride count from acceleration data or a digital pedometer; and adding the bias offset to the estimated stride length.

In some embodiments, a method comprises: obtaining, with at least one processor, sensor data from a wearable device worn on a wrist of a user; deriving, with the at least one processor, features from the sensor data; estimating, with the at least one processor, a first stride length using an estimation model that takes the features and user height as input; and calibrating, with the at least one processor, the first stride length of the user; obtaining, with the at least one processor, cadence and speed of the user; determining, with the at least one processor, a second estimated stride length of the user based on the cadence and speed; combining, with the at least one processor, the first estimated stride length and the second estimated stride length to get a final estimated stride length of the user.

In some embodiments, a system comprises: at least one processor; memory storing instructions that when executed by the at least one processor, cause the at least one processor to perform the methods recited above.

In some embodiments, a non-transitory, computer-readable storage medium having stored thereon instructions that when executed by the at least one processor, causes the at least one processor to perform the methods recited above.

Particular embodiments described herein provide one or more of the following advantages. The disclosed embodiments provide an estimate of stride length at the wrist that is personalized to the user, and therefore can provide more accurate fitness metrics (e.g., more accurate distance traveled), require only a wrist-worn sensor and does not require GPS (e.g., can run in environments where GPS is low quality, such as urban canyons and trails with foliage).

The details of one or more implementations of the subject matter are set forth in the accompanying drawings and the description below. Other features, aspects and advantages of the subject matter will become apparent from the description, the drawings, and the claims.

### BRIEF DESCRIPTION OF THE DRAWINGS

FIG. 1A is a system for estimating form-based stride length, according to some embodiments.

FIG. 1B is an alternative system for blending form-based stride length and speed-based stride length estimates, according to some embodiments.

FIG. 2A is a flow diagram of a process for estimating stride length, according to some embodiments.

FIG. 2B is a flow diagram of an alternative process for blending form-based stride length and speed-based stride length estimates, according to some embodiments.

FIG. 3 is example system architecture implementing the features and operations described in reference to FIGS. 1-2.

### DETAILED DESCRIPTION

### Example Systems

Technical materials typically refer to “stride length” as “step length,” whereas those authors use “stride” to mean two steps. As used herein, “stride length” is the length of a single step (“heel print to heel print”). It is a measure of running form that is related to running efficiency and injury risk. Runners are typically coached to avoid “over-striding,” which means running with a stride that places the foot ahead of the center of mass at initial foot contact. Over-striding is inefficient because it subjects the runner to unnecessary braking forces when the foot is in contact with the ground, and these braking forces act opposite the direction of travel.

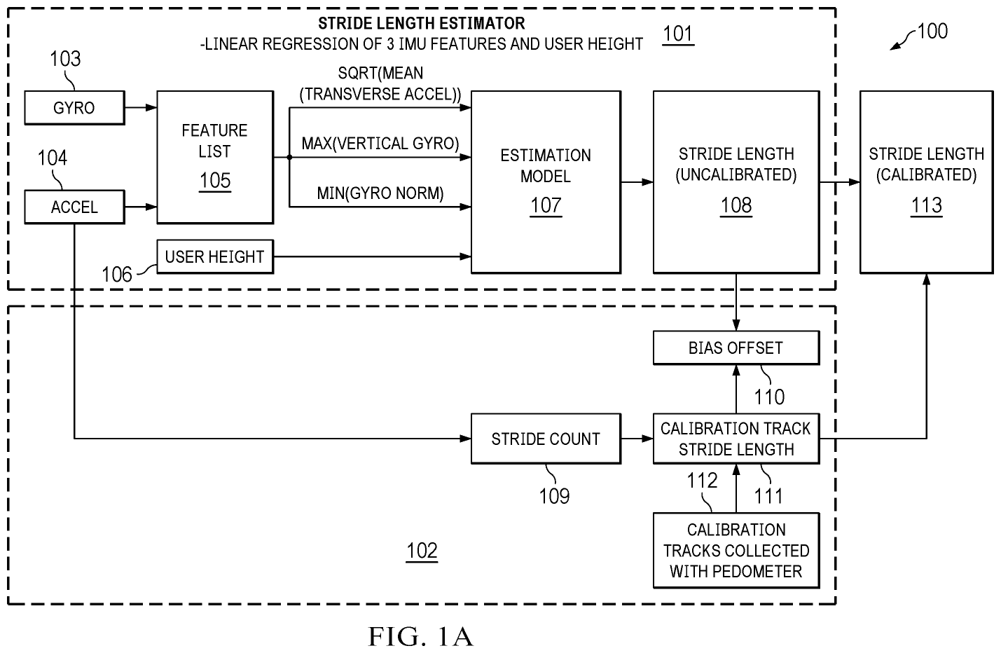

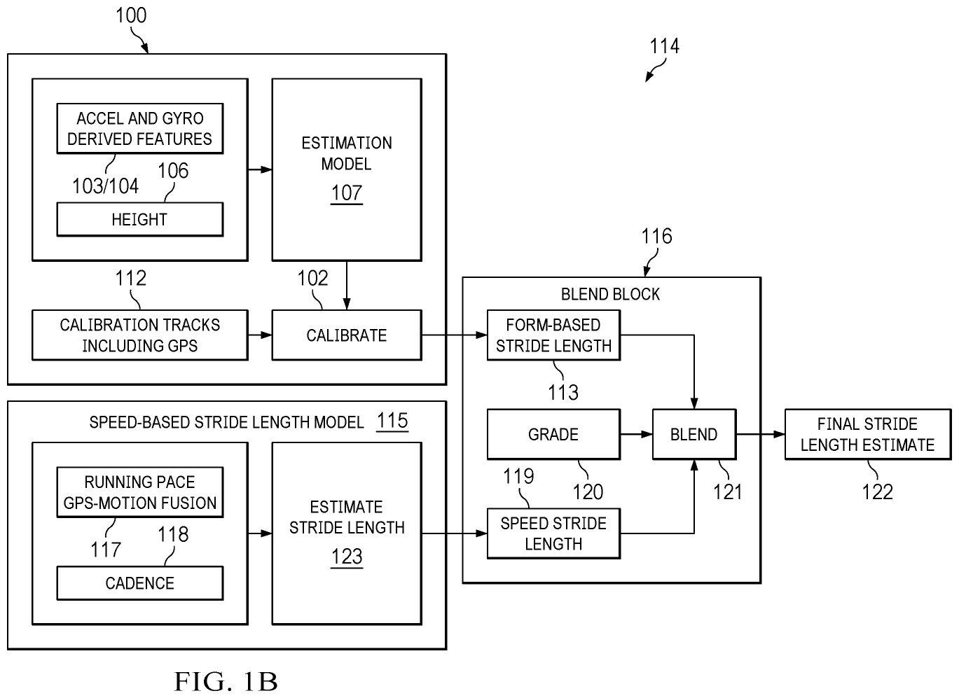

FIG. 1A is a system 100 for estimating form-based stride length, according to some embodiments. System 100 includes stride length estimator 101 and calibrator 102. System 100 can be implemented on a wearable device, such as a smartwatch or fitness band. Stride length estimator 101 takes as input a rotation rate vector from 3-axis gyro sensor 103 and an acceleration vector from 3-axis accelerometer sensor 104. Feature list 105 is generated by deriving features from the rotation rate and acceleration vectors. In some embodiments, feature list 105 includes the square root of the mean of transverse acceleration, maximum vertical rotation rate and minimum normalized rotation rate (magnitude). Other embodiments may have more or fewer features.

Feature list 105 is input into estimation model 107 with user height 106, which can be provided by a user through, for example, a graphical user interface (GUI). In some embodiments, estimation model 107 is a linear regression model, described more fully below. The output of estimation model 107 is uncalibrated stride length estimate 108. Bias offset 110 is used to adjust (e.g., multiplicative, or additive bias offsets) uncalibrated stride length measurement 108 to produce calibrated stride length estimate 113.

Calibrator 102 takes as input stride count 109 and calibration tracks 112. A “calibration track,” as used herein, is data collected (e.g., collected from a GPS receiver) during a period of time that meets optimal conditions for estimation of speed and cadence. Stride count 109 is obtained from the acceleration vector (e.g., using zero crossings, peak to peak analysis), or taken from a digital pedometer of the wearable device. Stride count 109 and calibration tracks are used to generate bias offset 110, which is added to uncalibrated stride length 108 to get calibrated stride length 113. In some embodiments, the calibration tracks are collected opportunistically during period of time meeting criteria for accurate speed and cadence estimation. The data associated with the calibration tracks (e.g., such GPS speed, GPS distance, cadence, grade) is stored on the mobile device. GPS distances from calibration tracks 112 can be divided by stride count 109 for the same time period to get a “truth” stride length, which is a stride length estimate suitable for use in calibration. Bias offset 110 is the difference between the true stride length and estimated stride length 108 over the same time period.

In the example shown, calibrator 102 calculates additive bias offset 110 using calibration tracks in a single bin (i.e., narrow range of speeds), intended to contain the user's typical running speed. The same bias offset is then applied to stride length estimates made during running at any speed. In some embodiments, when calibration tracks are available across various speeds, a linear fit can be made to the error and/or an alternate functional form may be used to calibrate stride length at the fastest and slowest running speeds.

### Calibration Track Requirements

In some embodiments, calibration tracks 112 have particular criteria to ensure acceptable results, which include one or more of consistent pedometer estimates and consistence GNSS (e.g., GPS) data. For example, pedometer estimates should be consistent when the user's arm is swinging unconstrained and should have low variability between subsequent measurements. Consistent GNSS data should be obtained GPS data from a single source (e.g., a smartwatch, companion device coupled to smartwatch) with horizontal accuracy below a specified threshold. Additionally, there should be concordance between average GNSS speed and total distance over total time and the average grade of the calibration track should be within a specified tolerance (e.g., +/−2% grade).

### Calibration Method and Extensions

In some embodiments where calibration tracks are within a narrow range of speeds that the user commonly runs, a single calibration constant can be derived by comparing raw stride length estimates to stride length derived from GNSS distance and step count (e.g., from the pedometer). In some embodiments, calibration tracks across a range of speeds are used to derive a calibration function dependent on speed that generates the calibration constant. In some embodiments, calibration tracks across a range of speeds and grades are used to derive a calibration function dependent on speed and grade that generates the calibration constant.

### Example Blended Stride Length Models

FIG. 1B is an alternative system 114 for blending the form-based stride length estimate computed in reference to FIG. 1A, and a speed-based stride length estimate, according to some embodiments. System 114 includes system 100 shown in FIG. 1A, speed-based stride length model 115 and blend block 116. Speed-based stride length model 115 takes as input running speed 117 and cadence 118 and estimates speed-based stride length 119. Blend block 116 takes form-based stride length 113, speed-based stride length estimate 119 and grade 120 as inputs and blends them to get final stride length estimate 122 of the user.

In some embodiments, speed-based stride length estimate 119 can be computed by dividing speed 117 by cadence 118. Grade can be calculated as elevation change (e.g., based on barometer data) divided by change in running speed 117 (change in horizontal speed). Speed can be obtained from a GPS or by integrating accelerations from accelerometers.

In some embodiments, the form-based stride length model 113 is used only when grade 120 satisfies a first condition (e.g., grade <x %), and the speed-based stride length model 115 is used only when grade 120 satisfies a second condition (e.g., grade >y %). In some embodiments, a convex combination of the form-based and speed-based stride estimates can be used across a transition region of moderate grade z (e.g., y<z<x), as shown in Equation [1]:

(1−α)f(t)+αg(t), [1]

where α is defined as:

$$α = \{ \begin{matrix}0 , & if \vert γ \vert \leq {G}_{1} \\ \frac{\vert γ \vert - {G}_{1}}{{G}_{2} - {G}_{1}} , & if {G}_{1} < \vert γ \vert < {G}_{2} \\ 1 , & if \vert γ \vert \geq {G}_{2}\end{matrix}$$

where f(t) is the form-based estimate of stride length, g(t) is the speed-based estimate of stride length, γ is the grade, and G₁ and G₂ are positive values that define the grade transition region, with G₂>G₁. α increases linearly from 0 to 1 as grade increases in the grade transition region from G₁ to G₂.

In some embodiments, blend block 116 is replaced with a concordance block that checks for consistency between the form-based stride length estimate 113 and speed-based stride length estimate 119 with no reference to grade 120. For example, when the two estimates are concordant, form-based stride length estimate 113 is used. Otherwise, final stride length estimate 122 is used.

In some embodiments, blend block 116 can blend form-based stride length 113 with speed-based stride length 119 using a weighted summation. For example, final stride length estimate 122 can be calculated as shown in Equation [2]:

stride_length_{final}=w₁*stridelength_{form}+w₂*stridelength_{speed}, [2]

where the weights w₁ and w₂ are determined empirically (e.g., w₁=0.5, w₂=0.5). In some embodiments, the estimates are averaged.

### Example Form-Based Model

In some embodiments, estimation model 107 uses a multivariate linear model (e.g., Lasso regression model) that is fitted using an L1 regularized loss function. The multivariable linear model is given by Equation [3]:

γ=β₀+β₁x₁+. . . +βₘxₘ, [3]

and the L1 regularized loss function is given by Equation [4]:

L(γ,\hat{γ})=Σᵢ₌₁ⁿ(γᵢ−\hat{γ}ᵢ)²+λ∥β∥₁, [4]

where \hat{γ}=Σⱼ₌₁^{m}βⱼxⱼ is the prediction made on the ith sample.

Table I below shows the base signal, the features selected and the source signal for deriving the features.

TABLE I
Base Signal/Selected Features/Source of Derivation

| Base Signal | Features Selected | Source of Derivation |
|---|---|---|
| NA | Height | User entry (e.g., through GUI) |
| User acceleration | Mean of L² norm of non- | Device Motion |
| | vertical component | (acceleration, rotation rate for |
| | | inertial frame) |
| Gyro rotation rate | Maximum of inertial frame | Device Motion |
| | vertical component minimum | (acceleration, rotation rate for |
| | of L² norm | inertial frame) |
| Tangential acceleration | NA | Arm swing-CoM decoupling |
| Centripetal acceleration | NA | Arm swing-CoM decoupling |
| Tangential velocity | NA | Arm swing-CoM decoupling |
| COM acceleration | NA | Arm swing-CoM decoupling |
| Arm swing rotation axis | NA | Arm swing-CoM decoupling |

The features listed in Table I are examples and not all the features may be used to estimate stride length. Other features may be used in addition to the features listed above.

In some embodiments, each base signal in Table I can be used to create 8 derived signals (3 device frame components, 3 inertial frame components, L2 norm of non-vertical signal, L2 norm of entire signal). Each derived signal can be aggregated in 6 ways (max, min, range, mean, standard deviation, area under curve) over periods demarcated by arm swing extrema. Arm swing extrema can be identified using peak to peak detection from the tangential acceleration signal derived from the arm swing-CoM decoupling. In some embodiments, features are aggregated over a full arm swing (backward and forward). In other embodiments, features are aggregated over backward swing only and forward swing only.

In some embodiments, arm swing-CoM decoupling is computed as follows. A centripetal acceleration estimator receives a window of sensor data including device motion (DM) rotation rate vector to and user acceleration vector u in body coordinates. The acceleration vector and rotation rate vector can be generated by, for example, 3-axis accelerometers and 3-axis gyros embedded in a wrist worn wearable device, such as a smartwatch. The window of sensor data is selected to include at least one stride cycle. The rotation rate vector to is input into principal component analysis (PCA) unit which estimates a primary rotation axis vector, Ω′. The rotation rate about the primary rotational axis is computed according to Equation [5]:

Ω=Ω′*(Ω′·ω). [5]

Tangential acceleration estimator receives DM user acceleration u and step frequency/cadence (e.g., form a digital pedometer) and estimates tangential acceleration (dv/dt), which is integrated to give tangential/transverse velocity v.

The centripetal acceleration a is calculated from the cross-product of Ω and v, as shown in Equation [6]:

α=Ωxν. [6]

The centripetal acceleration a is then subtracted from the user acceleration u to give a modified user acceleration α′=u-a with the estimated centripetal acceleration removed. This vector is input into an arm swing decoupler, which applies a bandpass filter to the input to decouple the arm swing acceleration component from the modified user acceleration. The output of arm swing decoupler is the CoM acceleration with the arm swing acceleration component removed. The CoM acceleration, tangential acceleration, centripetal acceleration, DM user acceleration and DM rotation rate are input features into estimation model 107.

### Example Processes

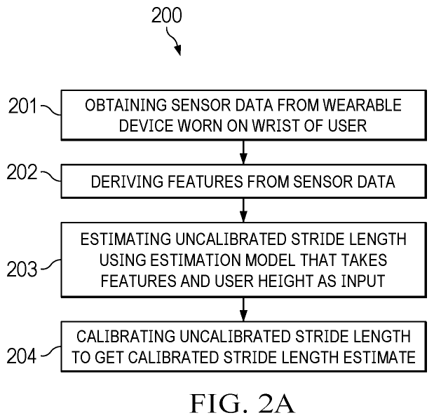

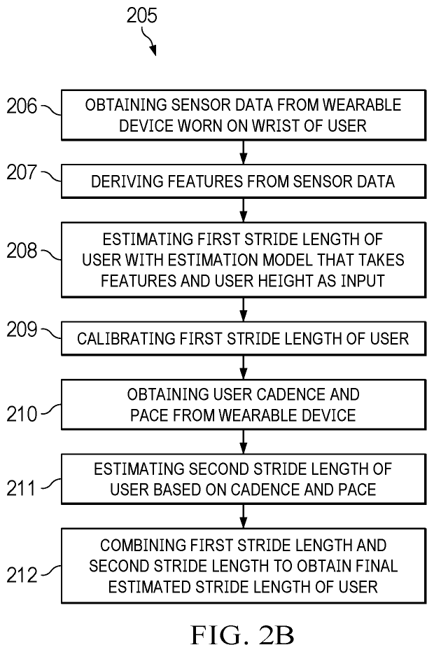

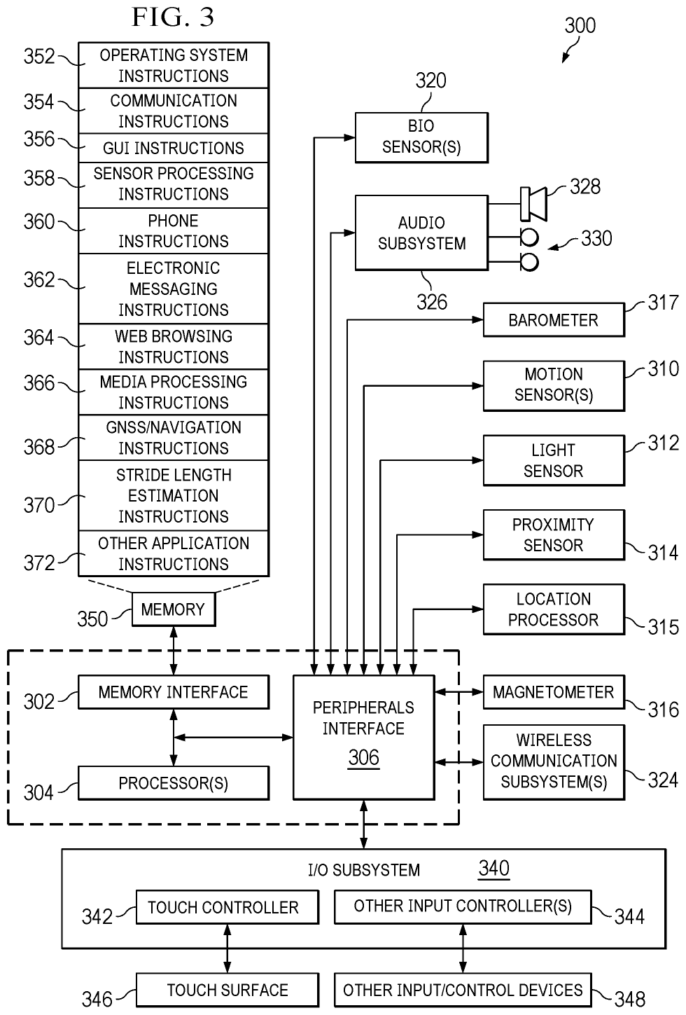

FIG. 2A is a flow diagram of a process for measuring form-based stride length, according to some embodiments. Process 200 can be implemented by, for example, using system architecture 300 described in reference to FIG. 3.

In some embodiments, process 200 includes: obtaining sensor data from a wearable device worn on a wrist of a user (201); deriving features from the sensor data (202); estimating stride length using an estimation model that takes the derived features and user height as input (203); and calibrating the stride length (204). Each of these steps were previously described in reference to FIG. 1A.

FIG. 2B is a flow diagram of an alternative process for blending form-based stride length and speed-based stride length, according to some embodiments. Process 205 can be implemented by, for example, using system architecture 300 described in reference to FIG. 3.

In some embodiments, process 205 includes: obtaining sensor data from a wearable device worn on a wrist of a user (206); deriving features from the sensor data (207); estimating a first stride length using an estimation model that takes the features and user height as input (208); calibrating the first stride length of the user (209); obtaining cadence and user speed of the user (210); determining a second stride length of the user based on the cadence and speed (211); combining the first stride length and the second stride length into a final estimated stride length of the user (212). Each of these steps were previously described in reference to FIGS. 1A and 1B.

### Exemplary System Architectures

FIG. 3 illustrates example system architecture 300 implementing the features and operations described in reference to FIGS. 1-2. Architecture 300 can include memory interface 302, one or more hardware data processors, image processors and/or processors 304 and peripherals interface 306. Memory interface 302, one or more processors 304 and/or peripherals interface 306 can be separate components or can be integrated in one or more integrated circuits. System architecture 300 can be included in any wearable device, including but not limited to: a smartwatch, fitness band, etc.

Sensors, devices, and subsystems can be coupled to peripherals interface 306 to provide multiple functionalities. For example, one or more motion sensors 310, light sensor 312 and proximity sensor 314 can be coupled to peripherals interface 306 to facilitate motion sensing (e.g., acceleration, rotation rates), lighting and proximity functions of the mobile device. Location processor 315 can be connected to peripherals interface 306 to provide geo-positioning. In some implementations, location processor 315 can be a GNSS receiver, such as the Global Positioning System (GPS) receiver. Electronic magnetometer 316 (e.g., an integrated circuit chip) can also be connected to peripherals interface 306 to provide data that can be used to determine the direction of magnetic North. Electronic magnetometer 316 can provide data to an electronic compass application. Motion sensor(s) 310 can include one or more accelerometers and/or gyros configured to determine change of speed and direction of movement. Barometer 317 can be configured to measure atmospheric pressure, which can be used to determine altitude. Biosensors 320 can include a heart rate sensor, such as a photoplethysmography (PPG) sensor.

Communication functions can be facilitated through wireless communication subsystems 324, which can include radio frequency (RF) receivers and transmitters (or transceivers) and/or optical (e.g., infrared) receivers and transmitters. The specific design and implementation of the communication subsystem 324 can depend on the communication network(s) over which a mobile device is intended to operate. For example, architecture 300 can include communication subsystems 324 designed to operate over a GSM network, a GPRS network, an EDGE network, a Wi-Fi™ network and a Bluetooth™ network. In particular, the wireless communication subsystems 324 can include hosting protocols, such that the mobile device can be configured as a base station for other wireless devices.

Audio subsystem 326 can be coupled to a speaker 328 and a microphone 330 to facilitate voice-enabled functions, such as voice recognition, voice replication, digital recording, and telephony functions. Audio subsystem 326 can be configured to receive voice commands from the user.

I/O subsystem 340 can include touch surface controller 342 and/or other input controller(s) 344. Touch surface controller 342 can be coupled to a touch surface 346. Touch surface 346 and touch surface controller 342 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch surface 346. Touch surface 346 can include, for example, a touch screen or the digital crown of a smart watch. I/O subsystem 340 can include a haptic engine or device for providing haptic feedback (e.g., vibration) in response to commands from processor 304. In an embodiment, touch surface 346 can be a pressure-sensitive surface.

Other input controller(s) 344 can be coupled to other input/control devices 348, such as one or more buttons, rocker switches, thumbwheel, infrared port, and USB port. The one or more buttons (not shown) can include an up/down button for volume control of speaker 328 and/or microphone 340. Touch surface 346 or other controllers 344 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 346; and a pressing of the button for a second duration that is longer than the first duration may turn power to the mobile device on or off. The user may be able to customize a functionality of one or more of the buttons. The touch surface 346 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 302 can be coupled to memory 350. Memory 350 can include high-speed random-access memory and/or non-volatile memory, such as one or more magnetic disk storage devices, one or more optical storage devices and/or flash memory (e.g., NAND, NOR). Memory 350 can store operating system 352, such as the iOS operating system developed by Apple Inc. of Cupertino, California. Operating system 352 may include instructions for handling basic system services and for performing hardware dependent tasks. In some implementations, operating system 352 can include a kernel (e.g., UNIX kernel).

Memory 350 may also store communication instructions 354 to facilitate communicating with one or more additional devices, one or more computers and/or one or more servers, such as, for example, instructions for implementing a software stack for wired or wireless communications with other devices, such as a sleep/wake tracking device. Memory 350 may include graphical user interface instructions 356 to facilitate graphic user interface processing; sensor processing instructions 358 to facilitate sensor-related processing and functions; phone instructions 360 to facilitate phone-related processes and functions; electronic messaging instructions 362 to facilitate electronic-messaging related processes and functions; web browsing instructions 364 to facilitate web browsing-related processes and functions; media processing instructions 366 to facilitate media processing-related processes and functions; GNSS/Location instructions 368 to facilitate generic GNSS and location-related processes and instructions; and instructions 370 that implement the features and processes described in reference to FIGS. 1 and 2. Memory 350 further includes application instructions 372 for performing various functions using, for example, estimating stride length previously described in reference to FIGS. 1 and 2.

Each of the above identified instructions and applications can correspond to a set of instructions for performing one or more functions described above. These instructions need not be implemented as separate software programs, procedures, or modules. Memory 350 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non- personal information available to the content delivery services, or publicly available information.

What is claimed is:

1. A method comprising:
obtaining, with at least one processor, sensor data from a wearable device worn on a wrist of a user;
deriving, with the at least one processor, features from the sensor data;
estimating, with the at least one processor, stride length using an estimation model that takes the features and user height as input; and
calibrating, with the at least one processor, the stride length.

2. The method of claim 1, wherein the sensor data includes acceleration and rotation rate, and the features include at least one of square root of the mean of transverse acceleration, maximum vertical rotation rate or minimum normalized rotation rate.

3. The method of claim 1, wherein calibrating comprises:
calculating a bias offset using distances from calibration tracks and stride count from acceleration data or digital pedometer; and
adding the bias offset to the estimated stride length.

4. A method comprising:
obtaining, with at least one processor, sensor data from a wearable device worn on a wrist of a user;
deriving, with the at least one processor, features from the sensor data;
estimating, with the at least one processor, a first stride length using an estimation model that takes the features and user height as input;
calibrating, with the at least one processor, the first stride length of the user;
obtaining, with the at least one processor, cadence, and speed of the user;
determining, with the at least one processor, a second stride length of the user based on the cadence and speed; and
combining, with the at least one processor, the first stride length and the second stride length into a final estimated stride length of the user.

5. The method of claim 4, wherein the sensor data includes acceleration and rotation rate, and the features include at least one of square root of the mean of transverse acceleration, maximum vertical rotation rate or minimum normalized rotation rate.

6. The method of claim 4, wherein calibrating comprises:
calculating a bias offset using distances from calibration tracks and stride count from acceleration data or digital pedometer; and
adding the bias offset to the estimated stride length.

7. A system comprising:
at least one processor;
memory storing instructions that when executed by the at least one processor, cause the at least one processor to perform operations comprising:
obtaining sensor data from a wearable device worn on a wrist of a user;
deriving features from the sensor data;
estimating stride length using an estimation model that takes the features and user height as input; and
calibrating the stride length.

8. The system of claim 7, wherein the sensor data includes acceleration and rotation rate, and the features include at least one of square root of the mean of transverse acceleration, maximum vertical rotation rate or minimum normalized rotation rate.

9. The system of claim 7, wherein calibrating comprises:
calculating a bias offset using distances from calibration tracks and stride count from acceleration data or digital pedometer; and
adding the bias offset to the estimated stride length.

10. A system comprising:
at least one processor;
memory storing instructions that when executed by the at least one processor, cause the at least one processor to perform operations comprising:
obtaining sensor data from a wearable device worn on a wrist of a user;
deriving features from the sensor data;
estimating a first stride length using an estimation model that takes the features and user height as input;
calibrating the first stride length of the user;
obtaining cadence and speed of the user;
determining a second stride length of the user based on the cadence and speed; and
combining the first stride length and the second stride length into a final estimated stride length of the user.

11. The system of claim 10, wherein the sensor data includes acceleration and rotation rate, and the features include at least one of square root of the mean of transverse acceleration, maximum vertical rotation rate or minimum normalized rotation rate.

12. The system of claim 10, wherein calibrating comprises:
calculating a bias offset using distances from calibration tracks and stride count from acceleration data or digital pedometer; and
adding the bias offset to the estimated stride length.

13. A non-transitory, computer-readable storage medium having stored thereon instructions that when executed by the at least one processor, causes the at least one processor to perform operations comprising:
obtaining sensor data from a wearable device worn on a wrist of a user;
deriving features from the sensor data;
estimating stride length using an estimation model that takes the features and user height as input; and
calibrating the stride length.

14. The non-transitory, computer-readable storage medium of claim 13, wherein the sensor data includes acceleration and rotation rate, and the features include at least one of square root of the mean of transverse acceleration, maximum vertical rotation rate or minimum normalized rotation rate.

15. The non-transitory, computer-readable storage medium of claim 13, wherein calibrating comprises:
calculating a bias offset using distances from calibration tracks and stride count from acceleration data or digital pedometer; and
adding the bias offset to the estimated stride length.

16. A non-transitory, computer-readable storage medium having stored thereon instructions that when executed by the at least one processor, causes the at least one processor to perform operations comprising:
obtaining sensor data from a wearable device worn on a wrist of a user;
deriving features from the sensor data;
estimating a first stride length using an estimation model that takes the features and user height as input;
calibrating the first stride length of the user;
obtaining cadence and speed of the user;
determining a second stride length of the user based on the cadence and speed; and
combining the first stride length and the second stride length into a final estimated stride length of the user.

17. The non-transitory, computer-readable storage medium of claim 16, wherein the sensor data includes acceleration and rotation rate, and the features include at least one of square root of the mean of transverse acceleration, maximum vertical rotation rate or minimum normalized rotation rate.

18. The non-transitory, computer-readable storage medium of claim 16, wherein calibrating comprises:
calculating a bias offset using distances from calibration tracks and stride count from acceleration data or digital pedometer; and
adding the bias offset to the estimated stride length.
